(19) United States
(12) Patent Application Publication
(10) Pub. No.: US 2025/0128079 A1
(43) Pub. Date: Apr. 24, 2025

(54) Likelihood Determination of Stimulation Provoked Side Effects Regions in Deep Brain Stimulation

(71) Applicant: Boston Scientific Neuromodulation Corporation, Valencia, CA (US)

(72) Inventor: León M. Juárez Paz, Berlin (DE)

(21) Appl. No.: 18/915,076

(22) Filed: Oct. 14, 2024

Related U.S. Application Data
(60) Provisional application No. 63/592,824, filed on Oct. 24, 2023.

(51) Int. Cl. A61N 1/372

(52) U.S. Cl. CPC A61N 1/37264; A61N 1/37247

(57) ABSTRACT

Methods and systems for assisting the programming of stimulation parameters for deep brain stimulation (DBS) for a subject patient are described. An accumulated database comprises data from a plurality of historical fitting/programming sessions. The data can include various stimulation parameter sets that were tried for patients in the database; data relating to the position of the electrode lead with respect to anatomical features the patients' brains, for example, with respect to the alignment of their subthalamic nucleus (STN); scores indicating the therapeutic effectiveness of the stimulation parameters and/or stimulation parameters that lead to side effects in the patients; and data relating to stimulation field models (SFMs) for the various stimulation parameter sets. The database may be used to predict stimulation parameter sets that are likely to be therapeutically effective or to cause side effects for the subject patient.

### CROSS REFERENCE TO RELATED APPLICATIONS

This is a non-provisional application of U.S. Provisional Patent Application Ser. No. 63/592,824, filed Oct. 24, 2023, which is incorporated herein by reference in its entirety, and to which priority is claimed.

### FIELD OF THE INVENTION

This application relates to deep brain stimulation (DBS), and more particularly, to methods and systems for optimizing DBS.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) context. DBS has been applied therapeutically for the treatment of neurological disorders, including Parkinson's Disease, essential tremor, dystonia, and epilepsy, to name but a few. Further details discussing the treatment of diseases using DBS are disclosed in U.S. Pat. Nos. 6,845,267, and 6,950,707.

Each of these neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise a handheld external control device to remotely instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects. For example, bilateral DBS of the subthalamic nucleus (STN) has been shown to provide effective therapy for improving the major motor signs of advanced Parkinson's disease, and although the bilateral stimulation of the subthalamic nucleus is considered safe, an emerging concern is the potential negative consequences that it may have on cognitive functioning and overall quality of life (see A. M. M. Frankemolle, et al., Reversing Cognitive-Motor Impairments in Parkinson's Disease Patients Using a Computational Modelling Approach to Deep Brain Stimulation Programming, Brain 2010; pp. 1-16). In large part, this phenomenon is due to the small size of the STN. Even with the electrodes located predominately within the sensorimotor territory, the electrical field generated by DBS is non-discriminately applied to all neural elements surrounding the electrodes, thereby resulting in the spread of current to neural elements affecting cognition. As a result, diminished cognitive function during stimulation of the STN may occur due to non-selective activation of non-motor pathways within or around the STN.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads, but are also distributed circumferentially around the neurostimulation leads as segmented electrodes, can be used.

To facilitate such selection, the clinician generally programs the external control device, and if applicable the neurostimulator, through a computerized programming system. This programming system can be a self-contained hardware/software system, or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters.

When electrical leads are implanted within the patient, the computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads, or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that best addresses the neurological disorder(s). There is a need for methods and systems that assist a clinician in determining optimum stimulation parameters for treating the patient.

### SUMMARY

Disclosed herein is a method for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising: receiving imaging data for the subject patient, using the imaging data to determine a position of at least one of the electrode leads with respect to at least one anatomical feature of the subject patient's brain, receiving accumulated data from a database, wherein the accumulated data comprises data from previous patients relating electrode lead position with respect to anatomical features of the previous patients' brains to stimulation parameters evoking side effects in the previous patients, and using the accumulated data and the imaging data to determine stimulation parameters for the subject patient. According to some embodiments, the at least one anatomical feature comprises a subthalamic nucleus (STN). According to some embodiments, the imaging data for the subject patient comprises preoperative magnetic resonance imaging (MRI) data and postoperative computed tomography (CT) and/or MRI data. According to some embodiments, the at least one anatomical feature of the subject patient's STN comprises one or more of a medial/lateral axis, an anterior/posterior axis, a medial border, a lateral border, an anterior border, and a posterior border. According to some embodiments, using the imaging data to determine a position of at least one of the electrode leads with respect to at least one anatomical feature of the subject patient's STN comprises using the imaging data to determine a 3-D model of the patient's STN and voxelizing the 3-D model. According to some embodiments, the method further comprises determining one or more axes of the subject patient's STN using the 3-D model. According to some embodiments, principal component analysis is used to determine the one or more axes. According to some embodiments, the accumulated data comprises indications of stimulation field model (SFM) radii corresponding to stimulation parameters providing therapeutic benefits in at least some of the previous patients. According to some embodiments, determining stimulation parameters for the subject patient comprises: receiving information indicative of a trial stimulation parameter set for the subject patient, determining an SFM for the trial stimulation parameter set, determining a SFM radius for the trial stimulation parameter set, and comparing the SFM radius for the trial stimulation parameter set to SFM radii from the accumulated data. According to some embodiments, the trial stimulation parameter set is automatically suggested using a stimulation optimization algorithm. According to some embodiments, using the accumulated data and the imaging data to determine stimulation parameters for the subject patient comprises: displaying on a graphical user interface (GUI): a representation of a search space indicative of potential trial stimulation parameter sets, and one or more likelihood maps derived from the accumulated data, wherein the one or more likelihood maps indicate trial stimulation parameter sets within the search space that are likely to evoke side effects in the subject patient. According to some embodiments, the one or more likelihood maps indicate SFM radii corresponding to trial stimulation parameter sets that are likely to evoke side effects for the subject patient.

Also disclosed herein it a system for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the system comprising: an external computing device comprising control circuitry configured to perform a method, the method comprising: receiving imaging data for the subject patient, using the imaging data to determine a position of at least one of the electrode leads with respect to at least one anatomical feature of the subject patient's subthalamic nucleus (STN), receiving accumulated data from a database, wherein the accumulated data comprises data from previous patients relating electrode lead position with respect to anatomical features of the previous patients' STNs to stimulation parameters evoking side effects in the previous patients, and using the accumulated data and the imaging data to determine stimulation parameters for the subject patient. According to some embodiments, the imaging data for the subject patient comprises preoperative magnetic resonance imaging (MRI) data and postoperative computed tomography (CT) and/or MRI data. According to some embodiments, the at least one anatomical feature of the subject patient's STN comprises one or more of a medial/lateral axis, an anterior/posterior axis, a medial border, a lateral border, an anterior border, and a posterior border. According to some embodiments, using the imaging data to determine a position of at least one of the electrode leads with respect to at least one anatomical feature of the subject patient's STN comprises using the imaging data to determine a 3-D model of the patient's STN and voxelizing the 3-D model. According to some embodiments, the method further comprises determining one or more axes of the subject patient's STN using the 3-D model. According to some embodiments, principal component analysis is used to determine the one or more axes. According to some embodiments, the accumulated data comprises indications of stimulation field model (SFM) radii corresponding to stimulation parameters providing therapeutic benefits in at least some of the previous patients. According to some embodiments, determining stimulation parameters for the subject patient comprises: receiving information indicative of a trial stimulation parameter set for the subject patient, determining an SFM for the trial stimulation parameter set, determining a SFM radius for the trial stimulation parameter set, and comparing the SFM radius for the trial stimulation parameter set to SFM radii from the accumulated data. According to some embodiments, the trial stimulation parameter set is automatically suggested using a stimulation optimization algorithm. According to some embodiments, using the accumulated data and the imaging data to determine stimulation parameters for the subject patient comprises: displaying on a graphical user interface (GUI): a representation of a search space indicative of potential trial stimulation parameter sets, and one or more likelihood maps derived from the accumulated data, wherein the one or more likelihood maps indicate trial stimulation parameter sets within the search space that are likely to evoke side effects in the subject patient. According to some embodiments, the one or more likelihood maps indicate SFM radii corresponding to trial stimulation parameter sets that are likely to evoke side effects for the subject patient.

Also disclosed herein is a method for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising: receiving accumulated data from a database, wherein the accumulated data comprises data from previous patients relating stimulation field model (SFM) information compiled from previous patients for a plurality of trial stimulation parameter sets to stimulation parameters evoking side effects in the previous patients, and using the accumulated data to determine stimulation parameters for the subject patient. According to some embodiments, the accumulated data comprises indications of stimulation field model (SFM) radii corresponding to stimulation parameters providing therapeutic benefits in at least some of the previous patients. According to some embodiments, determining stimulation parameters for the subject patient comprises: receiving information indicative of a trial stimulation parameter set for the subject patient, determining an SFM for the trial stimulation parameter set, determining a SFM radius for the trial stimulation parameter set, and comparing the SFM radius for the trial stimulation parameter set to SFM radii from the accumulated data. According to some embodiments, the trial stimulation parameter set is automatically suggested using a stimulation optimization algorithm. According to some embodiments, using the accumulated data and the imaging data to determine stimulation parameters for the subject patient comprises: displaying on a graphical user interface (GUI): a representation of a search space indicative of potential trial stimulation parameter sets, and one or more likelihood maps derived from the accumulated data, wherein the one or more likelihood maps indicate trial stimulation parameter sets within the search space that are likely to evoke side effects in the subject patient. According to some embodiments, the one or more likelihood maps indicate SFM radii corresponding to trial stimulation parameter sets that are likely to evoke side effects for the subject patient.

Also disclosed herein is a system for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the system comprising: an external computing device comprising control circuitry configured to perform a method, the method comprising: receiving accumulated data from a database, wherein the accumulated data comprises data from previous patients relating stimulation field model (SFM) information compiled from previous patients for a plurality of trial stimulation parameter sets to stimulation parameters evoking side effects in the previous patients, and using the accumulated data to determine stimulation parameters for the subject patient. According to some embodiments, the accumulated data comprises indications of stimulation field model (SFM) radii corresponding to stimulation parameters providing therapeutic benefits in at least some of the previous patients. According to some embodiments, determining stimulation parameters for the subject patient comprises: receiving information indicative of a trial stimulation parameter set for the subject patient, determining an SFM for the trial stimulation parameter set, determining a SFM radius for the trial stimulation parameter set, and comparing the SFM radius for the trial stimulation parameter set to SFM radii from the accumulated data. According to some embodiments, the trial stimulation parameter set is automatically suggested using a stimulation optimization algorithm. According to some embodiments, using the accumulated data and the imaging data to determine stimulation parameters for the subject patient comprises: displaying on a graphical user interface (GUI): a representation of a search space indicative of potential trial stimulation parameter sets, and one or more likelihood maps derived from the accumulated data, wherein the one or more likelihood maps indicate trial stimulation parameter sets within the search space that are likely to evoke side effects in the subject patient. According to some embodiments, the one or more likelihood maps indicate SFM radii corresponding to trial stimulation parameter sets that are likely to evoke side effects for the subject patient.

Also disclosed herein is a method for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising: receiving accumulated data from a database, wherein the accumulated data comprises: trial stimulation parameter sets provided to previous patients, stimulation field model (SFM) information for the trial stimulation parameter sets, and information indicative of the therapeutic effectiveness of the trial stimulation parameter sets for the previous patients, using the accumulated data to predict stimulation parameters that are likely to be therapeutic for the subject patient. According to some embodiments, the method further comprises updating the accumulated database based on the therapeutic effectiveness of the predicted stimulation parameters.

Also disclosed herein is a non-volatile computer readable medium comprising instructions for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, wherein the non-volatile computer readable medium comprises instructions, which when executed on a computing device, configure the computing device to perform a method comprising: receiving imaging data for the subject patient, using the imaging data to determine a position of at least one of the electrode leads with respect to at least one anatomical feature of the subject patient's subthalamic nucleus (STN), receiving accumulated data from a database, wherein the accumulated data comprises data from previous patients relating electrode lead position with respect to anatomical features of the previous patients' STNs to stimulation parameters evoking side effects in the previous patients, and using the accumulated data and the imaging data to determine stimulation parameters for the subject patient. According to some embodiments, the imaging data for the subject patient comprises preoperative magnetic resonance imaging (MRI) data and postoperative computed tomography (CT) and/or MRI data. According to some embodiments, the at least one anatomical feature of the subject patient's STN comprises one or more of a medial/lateral axis, an anterior/posterior axis, a medial border, a lateral border, an anterior border, and a posterior border. According to some embodiments, using the imaging data to determine a position of at least one of the electrode leads with respect to at least one anatomical feature of the subject patient's STN comprises using the imaging data to determine a 3-D model of the patient's STN and voxelizing the 3-D model. According to some embodiments, the method further comprises determining one or more axes of the subject patient's STN using the 3-D model. According to some embodiments, principal component analysis is used to determine the one or more axes. According to some embodiments, the accumulated data comprises indications of stimulation field model (SFM) radii corresponding to stimulation parameters providing therapeutic benefits in at least some of the previous patients. According to some embodiments, determining stimulation parameters for the subject patient comprises: receiving information indicative of a trial stimulation parameter set for the subject patient, determining an SFM for the trial stimulation parameter set, determining a SFM radius for the trial stimulation parameter set, and comparing the SFM radius for the trial stimulation parameter set to SFM radii from the accumulated data. According to some embodiments, the trial stimulation parameter set is automatically suggested using a stimulation optimization algorithm. According to some embodiments, using the accumulated data and the imaging data to determine stimulation parameters for the subject patient comprises: displaying on a graphical user interface (GUI): a representation of a search space indicative of potential trial stimulation parameter sets, and one or more likelihood maps derived from the accumulated data, wherein the one or more likelihood maps indicate trial stimulation parameter sets within the search space that are likely to evoke side effects in the subject patient. According to some embodiments, the one or more likelihood maps indicate SFM radii corresponding to trial stimulation parameter sets that are likely to evoke side effects for the subject patient.

The invention may also reside in the form of a programed external device, such as a clinician programmer or other computing device (via its control circuitry) for carrying out the above methods, a programmed implantable pulse generator (IPG) or external trial stimulator (ETS) (via its control circuitry) for carrying out the above methods, a system including a programmed external device and IPG or ETS for carrying out the above methods, or as a computer-readable media for carrying out the above methods stored in an external device or IPG or ETS. The invention may also reside in one or more non-transitory computer-readable media comprising instructions, which when executed by a processor of a machine configure the machine to perform any of the above methods.

### BRIEF DESCRIPTION OF THE DRAWINGS

FIG. 1A shows an Implantable Pulse Generator (IPG).

FIG. 1B shows a percutaneous lead having split-ring electrodes.

FIGS. 2A and 2B show an example of stimulation pulses (waveforms) producible by the IPG or by an External Trial Stimulator (ETS).

FIG. 3 shows an example of stimulation circuitry useable in the IPG or ETS.

FIG. 4 shows an ETS environment useable to provide stimulation before implantation of an IPG.

FIG. 5 shows various external devices capable of communicating with and programming stimulation in an IPG or ETS.

FIG. 6 illustrates an embodiment of a user interface (UI) for programming stimulation.

FIG. 7 illustrates an embodiment of a system for optimizing stimulation for DBS.

FIG. 8 illustrates a method of optimizing stimulation for DBS.

FIG. 9 illustrates a workflow for determining an orientation of an electrode lead with respect to anatomical features of a patient's STN.

FIG. 10 illustrates a cross section of an electrode lead.

FIGS. 11A and 11B show examples of determining distances between an electrode lead and anatomical features of a patient's STN.

FIGS. 12A and 12B show determined distances between an electrode lead and anatomical features of a patient's STN.

FIG. 13 shows an embodiment of a database cataloguing distances between an electrode lead and anatomical features of a patient's STN for a plurality of patients.

FIG. 14 shows determining a location of a stimulation field model (SFM) with respect to anatomical features of a patient's STN.

FIG. 15 shows a GUI for optimizing stimulation parameters for DBS for a patient, including likelihood models.

FIGS. 16A and 16B show radii for SFMs of parameter sets that are therapeutic and parameter sets leading to side effects, respectively.

FIG. 17 shows target areas and avoidance regions.

FIG. 18 shows a GUI having overlaid avoidance regions.

### DETAILED DESCRIPTION

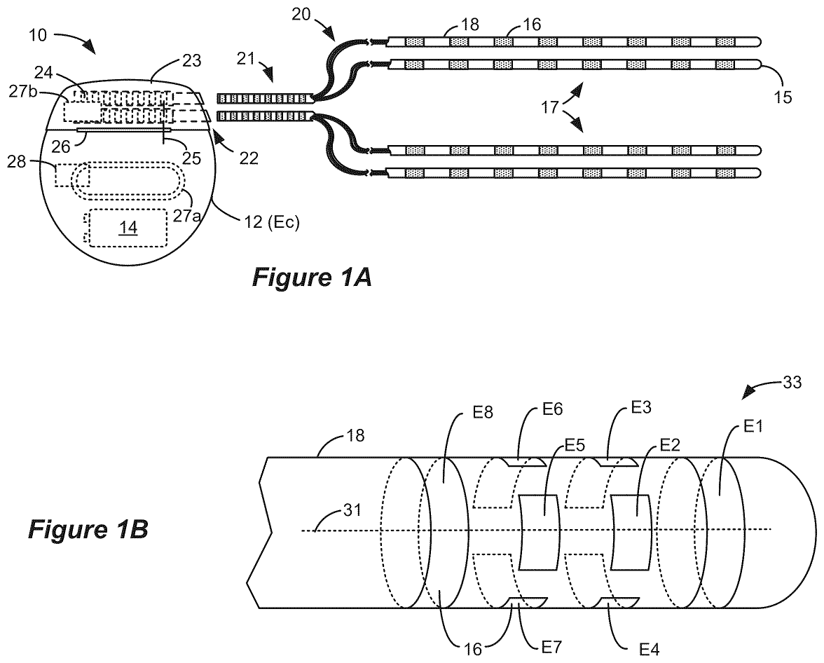

A DBS system typically includes an Implantable Pulse Generator (IPG) 10 shown in FIG. 1A. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more electrode leads 15 can be used having ring-shaped electrodes 16 carried on a flexible body 18.

In yet another example shown in FIG. 1B, an electrode lead 33 can include one or more split-ring electrodes. In this example, eight electrodes 16 (E1-E8) are shown. Electrode E1 at the distal end of the lead and electrode E8 at a proximal end of the lead comprise ring electrodes spanning 360 degrees around a central axis of the lead 33. In some embodiments, the electrode E1 may be a “bullet tip” electrode, meaning that it can cover the tip of the electrode lead. Electrodes E2, E3, and E4 comprise split-ring electrodes, each of which are located at the same longitudinal position along the central axis 31, but with each spanning less than 360 degrees around the axis. For example, each of electrodes E2, E3, and E4 may span 90 degrees around the axis 31, with each being separated from the others by gaps of 30 degrees. Electrodes E5, E6, and E7 also comprise split-ring electrodes, but are located at a different longitudinal position along the central axis 31 than are split ring electrodes E4, E2, and E3. As shown, the split-ring electrodes E2-E4 and E5-E7 may be located at longitudinal positions along the axis 31 between ring electrodes E1 and E8. However, this is just one example of a lead 33 having split-ring electrodes. In other designs, all electrodes can be split-ring, or there could be different numbers of split-ring electrodes at each longitudinal position (i.e., more or less than three), or the ring and split-ring electrodes could occur at different or random longitudinal positions, etc.

Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 15, and thus the header 23 may include a 2×2 array of eight-electrode lead connectors 22. However, the type and number of leads, and the number of electrodes, in an IPG is application-specific and therefore can vary. The conductive case 12 can also comprise an electrode (Ec).

In a DBS application, as is useful in the treatment of tremor in Parkinson's disease for example, the IPG 10 is typically implanted under the patient's clavicle (collarbone). Lead wires 20 are tunneled through the neck and the scalp and the electrode leads 15 (or 33) are implanted through holes drilled in the skull and positioned for example in the subthalamic nucleus (STN).

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Bluetooth Low Energy (BLE), as described in U.S. Patent Publication 2019/0209851, Zigbee, WiFi, MICS, and the like.

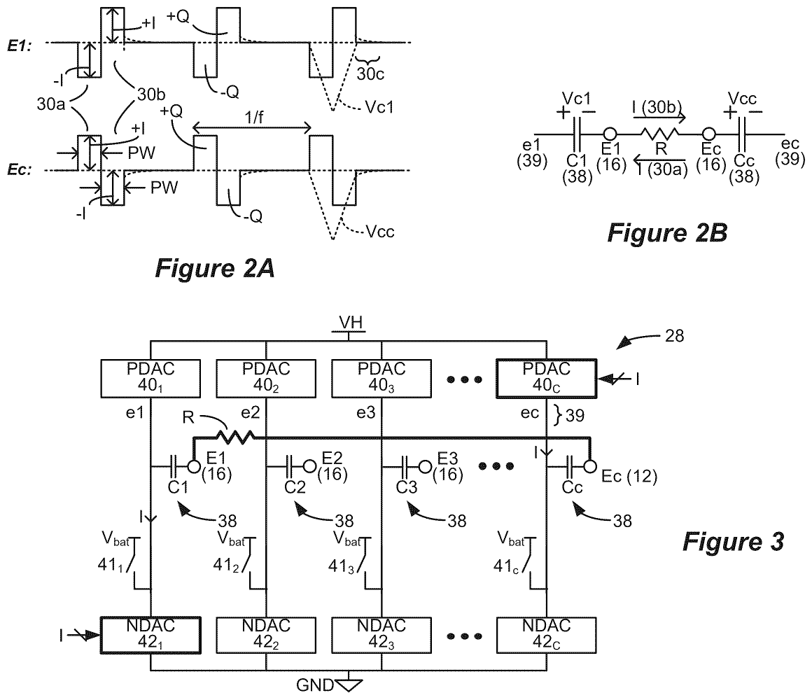

Stimulation in IPG 10 is typically provided by pulses each of which may include a number of phases such as 30a and 30b, as shown in the example of FIG. 2A. In the example shown, such stimulation is monopolar, meaning that a current is provided between at least one selected lead-based electrode (e.g., E1) and the case electrode Ec 12. Stimulation parameters typically include amplitude (current I, although a voltage amplitude V can also be used); frequency (f); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude-I from the tissue. The case electrode Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a) to ensure that the net current injected into the tissue is zero. The polarity of the currents at these electrodes can be changed: Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40ᵢ and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ci 39. Each electrode node Ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current through a patient's tissue, R, hopefully with good therapeutic effect. In the example shown, and consistent with the first pulse phase 30a of FIG. 2A, electrode E1 has been selected as a cathode electrode to sink current from the tissue R and case electrode Ec has been selected as an anode electrode to source current to the tissue R. Thus PDAC 40c and NDAC 421 are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40ᵢ and the electrode nodes ci 39, and between the one or more NDACs 42ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796. The stimulation circuitries described herein provide multiple independent current control (MICC) (or multiple independent voltage control) to guide the estimate of current fractionalization among multiple electrodes and estimate a total amplitude that provide a desired strength. In other words, the total anodic (or cathodic) current can be split among two or more electrodes and/or the total cathodic current can be split among two or more electrodes, allowing the stimulation location and resulting field shapes to be adjusted. For example, a “virtual electrode” may be created at a position between two physical electrodes by fractionating current between the two electrodes.

Much of the stimulation circuitry 28 of FIG. 3, including the PDACs 40ᵢ and NDACs 42ᵢ, the switch matrices (if present), and the electrode nodes eᵢ 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ci 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blocking capacitors Cl and Cc associated with the electrodes E1 and Ec used to produce the current, giving rise to voltages Vc1 and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors Cl and Cc is actively recovered, and thus voltages Vc1 and Vcc increase and return to 0V at the end of the second pulse phase 30b.

To recover all charge by the end of the second pulse phase 30b of each pulse (Vc1=Vcc=0V), the first and second phases 30a and 30b are charged balanced at each electrode, with the first pulse phase 30a providing a charge of −Q(−I*PW) and the second pulse phase 30b providing a charge of +Q(+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude (|I|) for each of the opposite-polarity pulse phases 30a and 30b. However, the pulse phases 30a and 30b may also be charged balanced at each electrode if the product of the amplitude and pulse widths of the two phases 30a and 30b are equal, or if the area under each of the phases is equal, as is known.

FIG. 3 shows that stimulation circuitry 28 can include passive recovery switches 41i, which are described further in U.S. Patent Application Publications 2018/0071527 and 2018/0140831. Passive recovery switches 41ᵢ may be attached to each of the electrode nodes ci 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41ᵢ. As shown in FIG. 3, the other end of the switches 41ᵢ not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery is less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

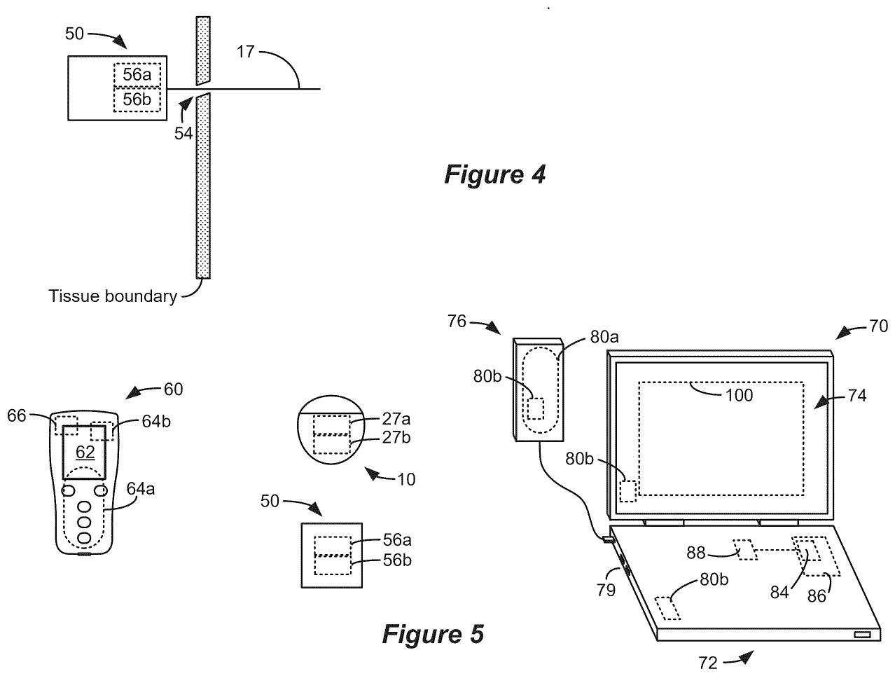

FIG. 4 shows an external trial stimulation environment that may precede implantation of an IPG 10 in a patient, for example, during the operating room to test stimulation and confirm the lead position. During external trial stimulation, stimulation can be tried on the implant patient to evaluate side-effect thresholds and confirm that the lead is not too close to structures that cause side effects. Like the IPG 10, the external trial stimulator (ETS) 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power. As the IPG may include a case electrode, an ETS may provide one or more connections to establish similar returns; for example, using patch electrodes. Likewise, the ETS may communicate with the clinician programmer (CP) so that the CP can process the data as described below.

FIG. 5 shows various external devices that can wirelessly communicate data with the IPG 10 or ETS 50, including a patient hand-held external controller 60, and a clinician programmer (CP) 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

External controller 60 can be as described in U.S. Patent Application Publication 2015/0080982 for example and may comprise a controller dedicated to work with the IPG 10 or ETS 50. External controller 60 may also comprise a general-purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 100 provided on the display 74 of the computing device 72. As one skilled in the art understands, the GUI 100 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 100, can also enable communications via antennas 80a or 80b to communicate stimulation parameters chosen through the GUI 100 to the patient's IPG 10.

The user interface of the external controller 60 may provide similar functionality because the external controller 60 can include similar hardware and software programming as the clinician programmer. For example, the external controller 60 includes control circuitry 66 similar to the control circuitry 88 in the clinician programmer 70 and may similarly be programmed with external controller software stored in device memory.

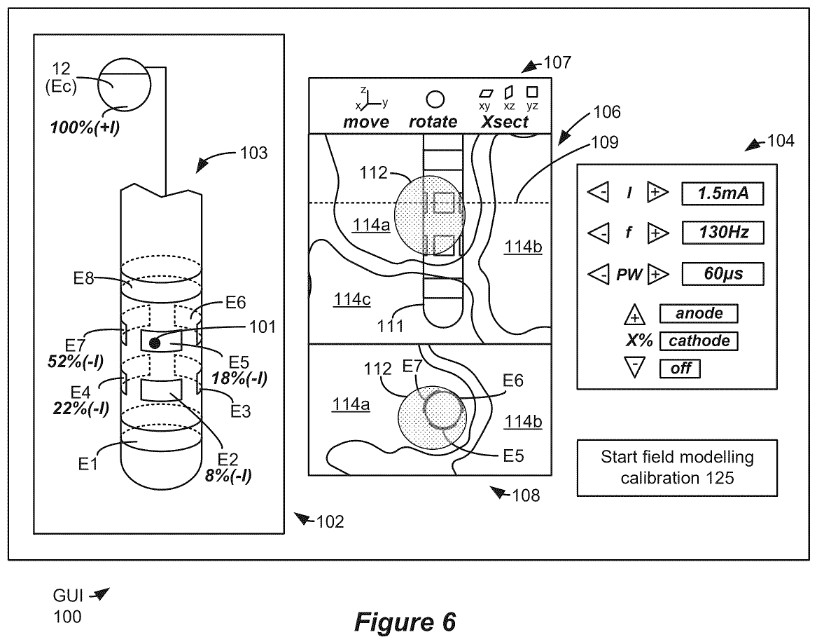

Particularly in the DBS context, it can be useful to provide a clinician with a visual indication of how stimulation selected for a patient will interact with the tissue in which the electrodes are implanted. This is illustrated in FIG. 6, which shows a Graphical User Interface (GUI) 100 operable on an external device capable of communicating with an IPG 10 or ETS 50. Typically, and as assumed in the description that follows, GUI 100 would be rendered on a clinician programmer 70 (FIG. 5), which may be used during surgical implantation of the leads, or after implantation when a therapeutically useful stimulation program is being chosen for a patient. However, GUI 100 could be rendered on a patient external programmer 60 (FIG. 5) or any other external device capable of communicating with the IPG 10 or ETS 50.

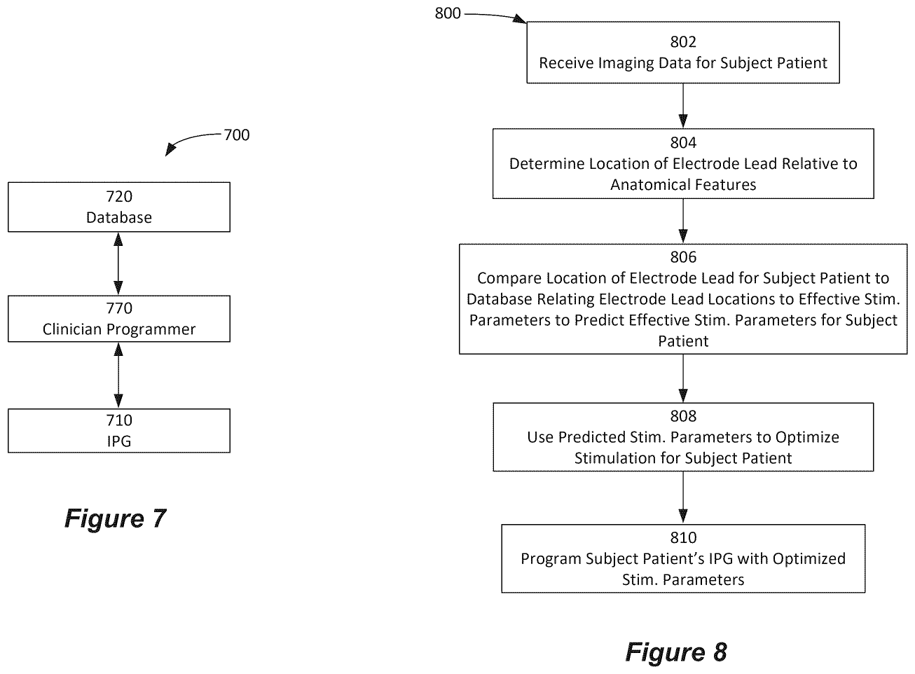

GUI 100 allows a clinician (or patient) to select the stimulation program that the IPG 110 or ETS 50 will provide and provides options that control sensing of innate or evoked responses, as described below. In this regard, the GUI 100 may include a stimulation parameter interface 104 where various aspects of the stimulation program can be selected or adjusted. For example, interface 104 allows a user to select the amplitude (e.g., a current I) for stimulation; the frequency (f) of stimulation pulses; and the pulse width (PW) of the stimulation pulses. Stimulation parameter interface 104 can be significantly more complicated, particularly if the IPG 10 or ETS 50 supports the provision of stimulation that is more complicated than a repeating sequence of pulses. Sec, e.g., U.S. Patent Application Publication 2018/0071513. Nonetheless, interface 104 is simply shown for simplicity in FIG. 7 as allowing only for amplitude, frequency, and pulse width adjustment. Stimulation parameter interface 104 may include inputs to allow a user to select whether stimulation will be provided using biphasic (FIG. 2A) or monophasic pulses, and to select whether passive charge recovery will be used, although again these details aren't shown for simplicity.

Stimulation parameter interface 104 may further allow a user to select the active electrodes—i.e., the electrodes that will receive the prescribed pulses. Selection of the active electrodes can occur in conjunction with a leads interface 102, which can include an image 103 of the one or more leads that have been implanted in the patient. Although not shown, the leads interface 102 can include a selection to access a library of relevant images 103 of the types of leads that may be implanted in different patients.

In the example shown in FIG. 6, the leads interface 102 shows an image 103 of a single split-ring lead 33 like that described earlier with respect to FIG. 1B. The leads interface 102 can include a cursor 101 that the user can move (e.g., using a mouse connected to the clinician programmer 70) to select an illustrated electrode 16 (e.g., E1-E8, or the case electrode Ec). Once an electrode has been selected, the stimulation parameter interface 104 can be used to designate the selected electrode as an anode that will source current to the tissue, or as a cathode that will sink current from the tissue. Further, the stimulation parameter interface 104 allows the amount of the total anodic or cathodic current +I or −I that each selected electrode will receive to be specified in terms of a percentage, X. For example, in FIG. 6, the case electrode 12 Ec is specified to receive X=100% of the current I as an anodic current +I. The corresponding cathodic current-I is split between electrodes E5 (0.18*−I), E7 (0.52*−I), E2 (0.08*−I), and E4 (0.22*−I). Thus, two or more electrodes can be chosen to act as anodes or cathodes at a given time using MICC (as described above), allowing the electric field in the tissue to be shaped. The currents specified at the selected electrodes can be those provided during a first pulse phase (if biphasic pulses are used), or during an only pulse phase (if monophasic pulses are used).

GUI 100 can further include a visualization interface 106 that can allow a user to view an indication of the effects of stimulation, such as a stimulation field model (SFM) 112 (also referred to herein as a volume of tissue activated (VTA)) formed using the selected stimulation parameters. The SFM 112 is formed by field modelling, for example, in the clinician programmer 70. The illustrated embodiment of the GUI 99 includes a selection option 125 for initiating such modeling. Only one lead is shown in the visualization interface 106 for simplicity, although again a given patient might be implanted with more than one lead. Visualization interface 106 provides an image 111 of the lead(s) which may be three-dimensional.

The visualization interface 106 preferably, but not necessarily, further includes tissue imaging information 114 taken from the patient, represented as three different tissue structures 114a, 114b and 114c in FIG. 6 for the patient in question, which tissue structures may comprise different areas of the brain for example. Such tissue imaging information may comprise a Magnetic Resonance Image (MRI), a Computed Tomography (CT) image or other type of image. Often, one or more images, such as an MRI, CT, and/or a brain atlas are scaled and combined in a single image model. As one skilled in the art will understand, the location of the lead(s) can be precisely referenced to the tissue structures 114i because the lead(s) are implanted using a stereotactic frame (not shown). This allows the clinician programmer 70 on which GUI 100 is rendered to overlay the lead image 111 and the SFM 112 with the tissue imaging information in the visualization interface 106 so that the position of the SFM 112 relative to the various tissue structures 114i can be visualized. The image of the patient's tissue may also be taken after implantation of the lead(s), or tissue imaging information may comprise a generic image pulled from a library which is not specific to the patient in question, in some embodiments.

The various images shown in the visualization interface 106 (i.e., the lead image 111, the SFM 112, and the tissue structures 114i) can be three-dimensional in nature, and hence may be rendered in the visualization interface 106 in a manner to allow such three-dimensionality to be better appreciated by the user, such as by shading or coloring the images, etc. Additionally, a view adjustment interface 107 may allow the user to move or rotate the images, using cursor 101 for example.

GUI 100 can further include a cross-section interface 108 to allow the various images to be seen in a two-dimensional cross section. Specifically, cross-section interface 108 shows a particular cross section 109 taken perpendicularly to the lead image 111 and through split-ring electrodes E5, E6, and E7. This cross section 109 can also be shown in the visualization interface 106, and the view adjustment interface 107 can include controls to allow the user to specify the plane of the cross section 109 (e.g., in XY, XZ, or YZ planes) and to move its location in the image. Once the location and orientation of the cross section 109 is defined, the cross-section interface 108 can show additional details. For example, the SFM 112 can allow the user to get a sense of the strength and reach of the stimulation at different locations. Although GUI 100 includes stimulation definition (102, 104) and imaging (108, 106) in a single screen of the GUI, these aspects can also be separated as part of the GUI 100 and made accessible through various menu selections, etc.

Especially in a DBS application, it is important that correct stimulation parameters be determined for a given patient. Improper stimulation parameters may not yield effective relief of a patient's symptoms, or may cause unwanted side effects. To determine proper stimulation, a clinician typically uses a GUI such as GUI 100 to try different combinations of stimulation parameters. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, the patient will typically present to the clinician's office to determine (or further refine) optimal stimulation parameters during a programming session.

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores(S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst).

Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score. U.S. Patent Application Publication 2021/0196956, which is incorporated herein by reference in its entirety, discusses determining which symptoms and/or side effects are most sensible to score for a given patient.

This disclosure relates to methods and systems for optimizing stimulation parameters for a DBS patient. In embodiments, quantitative objective information relating the position of the electrode lead with respect to anatomical features of the patient's STN are used to predict stimulation parameters that should be effective for the patient. FIG. 7 illustrates a simplified overview 700 of a system for performing aspects of the disclosed methods. The illustrated system comprises a clinician programmer (CP) 770, which may include the features described above with respect to the CP 70 (FIG. 5). The CP 770 may comprise control circuitry configured perform aspects of the disclosed methods. According to some embodiments, the CP 770 comprises non-transitory computer readable computer code, which when executed by the CP 770, configures the CP for performing aspects of the disclosed methods. The CP 770 may be configured to communicate an IPG 710, which may include the features described above with respect to the IPG 10 (FIGS. 1A and 5). The CP 770 may also be configured with access to a database 720. The features of the database 720 will be apparent based on the following discussion. The database 720 may be comprised within the CP 770, for example, within computer-readable storage. Alternatively (or additionally), aspects of the database 720 may be configured external to the CP 770, for example, on a local or remote external server. In such cases, the CP 770 may communicate with the database 720, for example, via an internet connection.

FIG. 8 illustrates a workflow 800 according to some aspects of the disclosure. It is assumed that one or more electrode leads have been implanted within the patient's brain. For example, the electrode leads may be implanted in, or near, a target brain structure, such as the patient's STN prior to executing the workflow 800. Other target brain structures for implantation may include the Globus Pallidus Internus, the Ventral Intermediate Nucleus, or any other target, as known in the art. Step 802 involves receiving imaging data for the subject patient. According to some embodiments, the imaging data may include pre-operative MRI data and post-operative CT scans. Step 804 involves determining the location of one or more of the electrode leads with respect to anatomical features of the patient's STN. According to some embodiments, the anatomical features may comprise various axes of the STN and/or various borders of the STN. For example, the anatomical features may comprise the medial border, the lateral border, the medio/lateral axis, the anterior border, the posterior border, the anterior/posterior axis, the superior border, the inferior border, and/or the inferior/superior axis or the like.

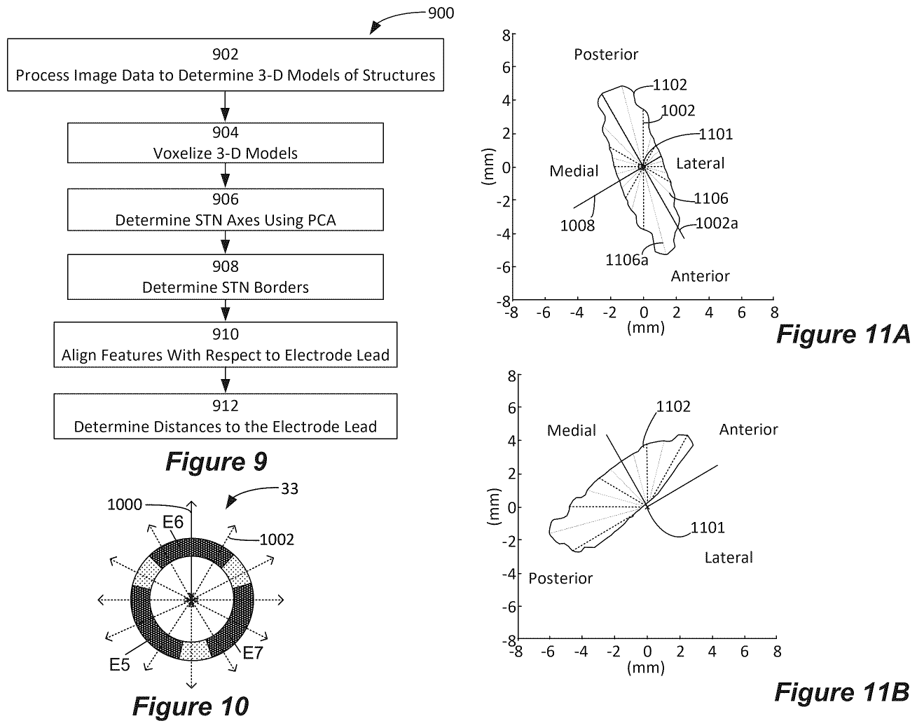

FIG. 9 illustrates an example of a workflow 900 for determining relevant anatomical features of the patient's STN and for determining the location of the electrode lead with respect to those anatomical features. Step 902 involve processing the image data to determine surfaces for the relevant brain structures, for example, the STN, the red nucleus (RN), Capsula Interna, Substantia Nigra, and the like. According to some embodiments, three dimensional models of the relevant structures may be generated from fusion of the MRI and CT images using individualized segmentation software auto-segmentation algorithms, as is known in the art. Example algorithms are included in commercial segmentation software, such as Brainlab Elements™, Brainlab, Germany. At step 904, the relevant 3-D models are voxelized, that is, the three dimensional structure is divided into volume elements, i.e., voxels.

At step 906, the voxelized model of the STN can be used to determine the axes of the STN. According to some embodiments, the determination of the STN axes mentioned in the next paragraph can also be done using the coordinates of the brain object's surface vertices. According to some embodiments, principal component analysis (PCA) is applied to the voxels to determine the medial/lateral, anterior/posterior, and/or superior/inferior axes of the STN. For example, the first PCA component may reflect the anterior/posterior axis, the second component may reflect the superior/inferior axis, and the third component may reflect the medial/lateral axis. Step 908 involves determining the relevant borders of the patient's STN using the voxeliztion and PCA described above.

Step 910 of the workflow 900 involves aligning the determined STN features with respect to the electrode lead. Aligning the STN features with respect to the electrode lead involves determining a relationship between the orientation of the STN within the patient (i.e., “patient space”) and the orientation of the STN features with respect to the electrode lead. Specifically, it may be desirable to quantify how the STN features, such as the axes, angularly align with the stimulation fields that the electrode lead is capable of producing.

FIG. 10 illustrates a cross section of an electrode lead 33 viewed down the central axis 31 of the lead having split ring electrodes (See, e.g., FIG. 1B). In some embodiments, the electrode lead 33 may have an orientation marker 1000, which informs the rotational positioning of the lead. For example, the electrode lead may be implanted so that the orientation marker 1000 is directed to the front of the patient's head. As mentioned above, the split ring (i.e., directional) electrodes may be used to form stimulation fields that emanate from the lead at different angles. However, in some embodiments, the resolution of the stimulation angle is limited. In the illustrated embodiment, the angular resolution at which stimulation can be controlled is limited to 30°, as indicated by the dashed lines 1002. Thus, stimulation fields may be controlled along the 30° lines and the angles can be measured relative to the orientation marker 1000. An aspect of step 910 of the workflow 900 (FIG. 9) therefore involves orienting or transforming the anatomical features of the STN, which may be in “patient space,” with respect to the orientation marker 1000 and the resolution lines 1002 of the electrode. This may be thought of as orienting the anatomical features of the STN with respect to “lead space.”

FIG. 11A illustrates one embodiment of aligning the medio/lateral axis and the anterior/posterior axes with an electrode lead in a situation when the electrode lead (positioned at the point 1101) has been implanted within the patient's STN 1102. In the illustration, the bold dashed lines 1002 represent the highest resolution at which angular stimulation can be controlled, as described above (which is 30°, in this example). In this example, upsampling may be used to increase the resolution at which PCA is used to determine the axes. The light dashed lines 1106 illustrate polar upsampling to provide 15° resolution for the axis determination. Other forms of upsampling, such as Cartesian upsampling may also be used. Notice that in FIG. 11A, the line 1106a corresponds to the longest component determined using polar upsampling. That line therefore represents the best estimate for the anterior/posterior axis. But also notice that the line 1106a is between angles at which the electrode lead can angularly resolve stimulation. The closest angularly resolvable line is 1002a. According to some embodiments, the line 1002a can simply be considered as the anterior/posterior axis. According to other embodiments, the alignment may be refined by projecting a component of the “truer axis” 1106a on the resolvable line 1002a to provide a more accurate estimation of the aligned distance of the electrode lead from the anterior and posterior borders. The same process can be used to align the electrode lead with medial/lateral axis and to estimate the distance of the electrode lead from the medial and lateral borders.

FIG. 11B shows an embodiment wherein the electrode lead (position 1101) is implanted near to, but not within the STN 1002. Similar techniques as described above can be used to either radially determine, or project the relevant axes with respect to the electrode lead alignment.

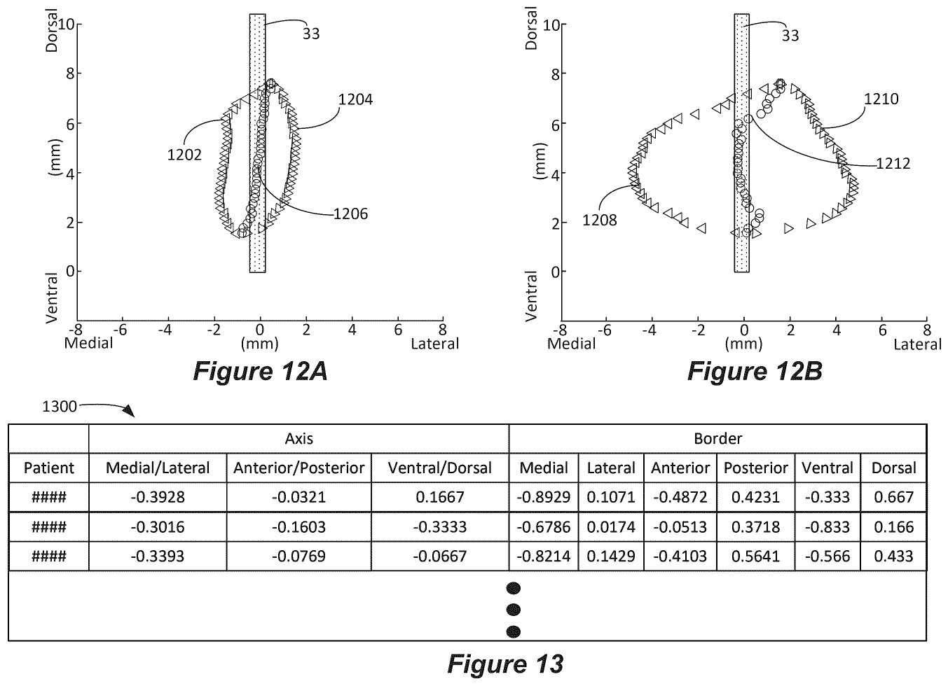

Referring again to FIG. 9, step 912 involves determining the distance of the electrode lead to the relevant anatomical structures of the STN, such as the axes and borders of the STN. FIG. 12A shows calculated distances from the electrode lead 33 from the medial border 1202, the lateral border 1204, and the medial/lateral axis 1206. FIG. 12B shows calculated distances from the electrode lead 33 from the anterior border 1208, the posterior border 1210, and the anterior/posterior axis 1212.

Once the location of the electrode lead relative to the anatomical features of the STN have been determined for the subject patient, those determined distances can be catalogued in a database. FIG. 13 illustrates an example of a database 1300 that is populated with data accumulated for a large number of patients. Assume that each of those patients have undergone fitting procedures whereby stimulation has been optimized for each of those patients. For each of those patients, stimulation parameters have been determined that has been shown to be therapeutically effective and/or that does not cause side effects for the patient. The orientation/distance information for each of the patients can then be correlated with the effective stimulation parameters and used as a predictive tool for optimizing stimulation for future subject patients. In other words, the orientation/distance measurements can be used as quantitative and objective criteria for predicting stimulation parameters that are likely to be therapeutic for the future subject patient. Accordingly, referring again to FIG. 8, step 806 involves comparing the location/distance information for the subject patient to a database relating accumulated orientation/location information to therapeutically effective stimulation parameters to predict stimulation parameters for the subject patient.

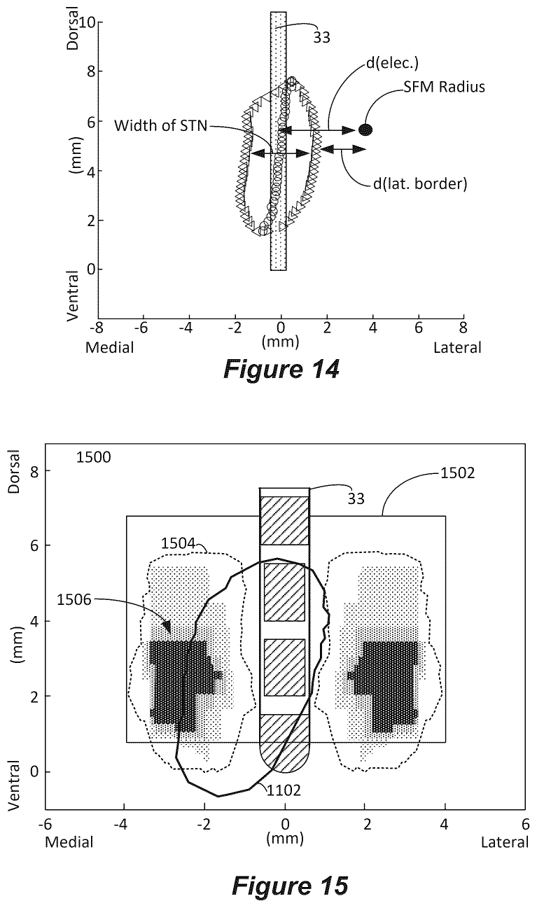

According to some embodiments, stimulation parameters may be optimized based on the radius of the stimulation field generated by the stimulation. FIG. 14 illustrates an electrode lead 33 shown in relation to the medial and lateral borders and the medial/lateral axis of an STN (similar to FIG. 12A). For a given set of stimulation parameters (e.g., amplitude, pulse width, frequency, duty cycle, etc.) stimulation field modeling can be used to predict the radius of the SFM. FIG. 14 illustrates a modeled SFM radius having a distance d (elect.) from the electrode 33. Since the distance between the electrode and the lateral border is known, the distance of the SFM radius and the lateral border d(lat. border) can be calculated for the particular set of stimulation parameters. Assume for a given patient, stimulation parameters yielding the SFM radius shown in FIG. 14 provides effective therapy and no side effects. It may be that other stimulation parameters yielding a smaller SFM radius (for example, stimulation parameters with a smaller amplitude) does not provide effective therapy. Likewise, perhaps stimulation parameters having a larger SFM radius (for example, stimulation parameters with a larger amplitude) causes side effects.

According to some embodiments, a database, such as the database 1300 (FIG. 13) can include information correlating the orientation/distance information for each of a number of patients with the effective stimulation parameters. The stimulation parameters may be expressed in terms of SFM radius, as discussed here. Alternatively, other stimulation parameter values, such as amplitude, stimulation field energy, frequency, pulse width, duty cycle, electrode configuration, or the like, may be collated in the database. The stimulation according to each of the trial parameter sets may be scored based on its effect for the patient on the basis of patient responses to the stimulation, as is known in the art. According to some embodiments, the patient responses may include one or more of speech, tremor, rigidity, finger tapping, toc tapping, bradykinesia, hypokinesia, agility posture, gate, postural stability, or the like.

According to some embodiments, when comparing the subject patient's electrode lead position with respect to the anatomical features of their STN to the values contained in the database 1300 for a plurality of patients, it may be desirable to normalize the distances with respect to the size of each individual's STN. This is because the STN of different patients may have different sizes. According to some embodiments, each set of data is normalized with respect to the maximum width of the STN (see FIG. 14). As other examples, the normalization could be done also based on the length or height of the STN (i.e., anterior-posterior or ventral-dorsal axes, respectively).

Referring again to FIG. 8, step 808 involves using the predicted stimulation parameters from the database to optimize stimulation for the subject patient. According to some embodiments, the database may simply indicate specific stimulation parameters to try, based on the determined orientation/distance information for the subject patient. According to other embodiments, the database may suggest a range of stimulation parameters that might be beneficial to the patient.

FIG. 15 illustrates a GUI 1500 configured for assisting a user when optimizing stimulation parameters for a subject patient using information from a database that correlates the electrode leads' position with respect to anatomical feature of the STN with stimulation parameters that are likely to provide effective therapy. As explained above, the database comprises historical data collected from a number of patients relating the location/distance information for those patients to stimulation parameters that were found to be effective. According to some embodiments, the GUI 1500 may be incorporated as a sub-feature of the GUI 100 discussed above (FIG. 6). The GUI 1500 includes a representation of the implanted electrode lead 33 and the patient's STN 1102.

In the illustrated example, assume that the stimulation parameters are being optimized based on the location of the SFM radius of the trial stimulation parameter sets. According to other embodiments, other aspects of the trial stimulation parameter sets may be used, such as individual parameter values (e.g., amplitude, frequency, pulse width, etc.). The illustrated GUI includes a border 1502 that defines a “search space” for trial SFM radii. In other words, the clinician will try various trial stimulation parameter sets yield SFM radii that fall within the space defined by the border 1502. It should be noted here, the optimization may be performed in a “brute force” way, whereby the clinician simply decides which parameter sets they wish to try within the search space. Alternatively, one or more optimization algorithms may be used to suggest parameter sets within the search space to try. For example, U.S. Patent Application No. 2022/0257950, the entire contents of which are incorporated herein by reference, describes a DBS optimization algorithm configured to suggest trial stimulation parameters within a search space.

The GUI 1500 features “likelihood maps” compiled from the historical data in the database. The likelihood maps provide indications of where effective stimulation parameters are likely to be found or where stimulation might cause side effects. In case of the side effects likelihood maps (or models), these may be generated based on the cumulative likelihood (starting from the lead and going in the direction of increased distance from it) of SFM radii leading to side effects. This likelihood might be estimated by e.g., bivariate histograms that consider the number of SFM radii falling within a combination of SFM location along the lead axis and the SFM radius. One example of likelihood maps is represented by the dashed lines 1504. Stimulation parameters that result in SFM radii within the space encompassed within 1504 are likely to be beneficial for the subject patient, based on the location of the electrode lead with respect to the subject patient's anatomical STN features. Such likelihood maps are derived by comparing the subject patient's lead orientation to those for the plurality of patients reflected in the database (typically after normalizing for STN size, as described above). The illustrated GUI 1500 also features heat maps 1506, which reflect locations in the search space where effective stimulation parameters are likely to be found. The heat maps may be based on histograms derived from the historic patient database. In the illustrated embodiment, the darker portions of the heat map indicate higher likelihoods of success. For example, the data in the database may indicate that when stimulation parameters provided SFM radii within the darker portions of the heat map, more of the patients responded well to the stimulation.

The likelihood models/maps (e.g., 1504 and 1506) effectively reduce the search space of parameters to interrogate during the optimization process. If the clinician is using a “brute force” method, the likelihood maps may indicate that the clinician should concentrate on parameter sets within the likelihood maps. Likewise, if an optimization algorithm (such as the algorithm described in the above-incorporated patent application) is being used, the search space available to the algorithm may be limited to the spaces indicated by the likelihood maps. Alternatively, parameter sets falling within the likelihood map may simply be weighted heavier within the optimization algorithm. In either case, the likelihood maps may reduce the number of parameter sets to interrogate and thereby reduce the amount of time required to optimize stimulation for the patient. Referring again to FIG. 8, step 810 involves programming the patient's IPG with the optimized stimulation parameters determined as described herein.

FIGS. 14 and 15, and the accompanying discussion above, explain how a database with accumulated information relating SFMs for stimulation parameter sets for a plurality of patients can be used to predict stimulation parameters are likely to provide a therapeutic benefits for a subject patient. A similar database with accumulated information relating SFMs for stimulation parameter sets for a plurality of historical patients can also be used to predict stimulation parameters that are likely to cause side effects. According to such an embodiment, a database may be constructed for a plurality of historical patients that have undergone a fitting procedure. During the fitting procedure, various trial stimulation parameter sets are applied to the patients. SFMs may be determined for each of the trial stimulation parameter sets. Trial stimulation parameter sets, and their corresponding SFM data, may be catalogued in a database. In particular, trial stimulation parameter sets that cause side effects may be noted and correlated with their SFM data, such as SFM radius information (or other features of the SFM). Such a side-effects database may be used when determining stimulation parameters for a subject patient to predict stimulation parameters that are likely to cause side-effects for the patient. In other words, such a database can be used to predict “avoidance regions” comprising parameter spaces defining stimulation parameters that are likely to lead to side effects in the patient.

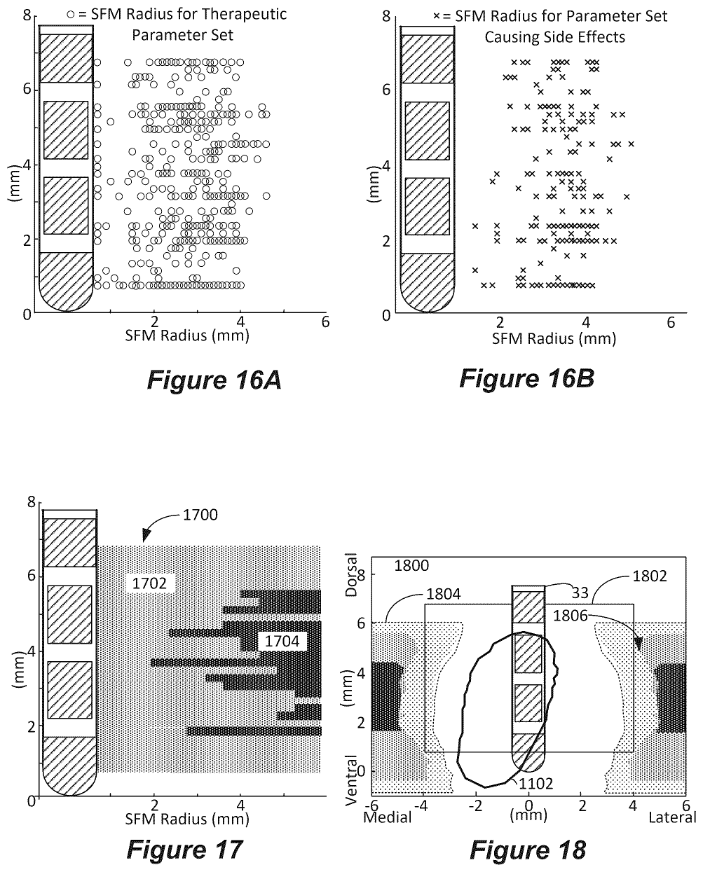

FIGS. 16A and 16B illustrate data contained in databases comprising SFMs for stimulation parameter sets accumulated for a plurality of patients that were found to be therapeutic (FIG. 16A) or were found to cause side effects (FIG. 16B). Specifically, the graphs relate the SFM radii for the various trial parameter sets to their therapeutic outcome. In FIG. 16A, each of the circles represent an SFM radius for stimulation parameters that was found to be therapeutic for a given patient in the database. In FIG. 16B, each of the x's represent an SFM radius for stimulation parameters that were found to cause side effects for a patient in the database.

FIG. 17 illustrates how the data shown in FIGS. 16A and 16B can be used to predict stimulation parameters that are expected to be therapeutic or that would be expected to cause side effects for a subject patient. A parameter space 1700 comprises SFM radii for various trial parameter sets. The parameter space 1700 comprises regions of SFM radii corresponding to parameter sets that are predicted to be therapeutic and not to lead to side effects 1702. The regions 1702 may be referred to as “likelihood regions.” The parameter space 1700 also comprises regions of SFM radii corresponding to parameter sets that are predicted to cause side effects 1704. The regions 1704 may be referred to as “avoidance regions.” When optimizing stimulation for a subject patient, a clinician would focus on trial stimulation parameter sets having SFM radii in the likelihood region instead of in the avoidance region.

It should be noted that that the database described with reference to FIGS. 16A, 16B, and 17 do not consider the location of the electrode lead with respect to anatomical features of the STN. Other embodiments may include determining the location of the electrode lead with respect to the STN anatomical features, as described above (see, e.g., FIG. 9). In such embodiments, the side effect database may comprise anatomical data, such as contained in the database 1300 (FIG. 13). For example, the side effect database may correlate SFM radii for stimulation parameters that cause side effects with information indicating the position of the electrode lead with respect to anatomical features of the STN, such as various axes of the STN (e.g., medial/lateral, anterior/posterior, and/or ventral/dorsal) and/or borders of the STN (e.g., medial, lateral, anterior, posterior, ventral, and/or dorsal borders). Historic information from such a side effect database can be projected onto a GUI, such as the GUI 1500 (FIG. 15) to aid in the programming of stimulation parameters for a subject patient. FIG. 18 shows an embodiment of a GUI 1800 that is similar to the GUI 1500 discussed above. The GUI 1800 includes a representation of the implanted electrode lead 33 and the patient's STN 1102. As with the embodiment of the GUI described above, the GUI 1800 includes a border 1802 that defines a “search space” for trial SFM radii. The GUI 1800 features “avoidance maps” compiled from the historical data in the database. The avoidance maps provide indications of stimulation parameters that are likely to cause side effects. One example of likelihood maps is represented by the dashed lines 1804. Stimulation parameters that result in SFM radii within the space encompassed within 1804 are deemed likely to cause side effects for the subject patient, based on the location of the electrode lead with respect to the subject patient's anatomical STN features. Such likelihood maps are derived by comparing the subject patient's lead orientation to those for the plurality of patients reflected in the database (typically after normalizing for STN size, as described above). The illustrated GUI 1800 also features heat maps 1806, which reflect locations in the search space where stimulation parameters that are likely to cause side effects may occur. The heat maps may be based on histograms derived from the historic patient side effect database. In the illustrated embodiment, the darker portions of the heat map indicate higher likelihoods of side effects. For example, the data in the database may indicate that when stimulation parameters provided SFM radii within the darker portions of the heat map, more of the patients experienced side effects in response to the stimulation. Thus, a clinician may seek to avoid stimulation parameters with SFM radii within those areas.

FIGS. 15, 17, and 18 show examples of how stimulation parameter information accumulated from a plurality of patients (and a plurality of STN hemispheres) relating to stimulation parameters that are shown to be therapeutic and/or to cause side effects can be used to inform the programming of stimulation parameters for a subject patient. As explained above, such databases may or may not include anatomical data estimating the position of the electrode lead(s) relative to anatomic features of the patients' STN. In either case, the database information may be used to generate likelihood models, which indicate parameter spaces where therapeutic parameters are likely to be obtained, and/or avoidance models, which indicate parameter spaces where parameters causing side effects are likely to occur. The examples described above focused primarily on using the radii of SFMs for the parameters accumulated in the historic database(s) as a basis for determining likelihood/avoidance spaces. Other aspects of the accumulated SFMs may also be used. For example, the volumes of the accumulated SFMs may be used to predict target and/or avoidance volumes for the stimulation of the subject patient. The target volume may predict a volume of the patient's brain where stimulation is likely to provide therapeutic benefit, whereas an avoidance volume may predict a volume where stimulation is likely to cause side effects.

As explained above, once the accumulated database has been used to predict target/likelihood regions and/or avoidance regions, those predicted regions can be used to facilitate the programming of stimulation parameters for a subject patient. According to some embodiments, programming optimization algorithms, such as those described in the incorporated U.S. Patent Application No. 2022/0257950 may be used to suggest trial stimulation parameters that are within the target/likelihood region and/or that avoid the avoidance regions. Alternatively, the clinician may simply choose, using “brute force,” various trial stimulation parameters based on the predicted target and avoidance regions. Following the application of each of the trial stimulation parameters, the impact of the stimulation on the patient may be scored.

According to some embodiments, it may be prudent to validate the veracity of the target and/or avoidance regions that are predicted based on the accumulated database(s). According to some embodiments, the final stimulation parameters that are ultimately found to provide the best stimulation for the patient may be compared to the target and/or avoidance regions predicted based on the accumulated database(s). If the final stimulation parameters agree well with the predicted target/avoidance regions, then that agreement may serve as a validation of the predicted regions. If there is a discrepancy between the final stimulation parameters and the predicted regions, that discrepancy may indicate that the target/avoidance regions predicted based on the accumulated database are not reliable. In such a case, one or more of the database entries may be tagged as being unreliable, in which case they may be ignored or de-emphasized in further programming sessions.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising:
receiving imaging data for the subject patient,
using the imaging data to determine a position of at least one of the electrode leads with respect to at least one anatomical feature of a brain structure of the subject patient's brain,
receiving accumulated data from a database, wherein the accumulated data comprises data from previous patients relating electrode lead position with respect to at least one anatomical features of a brain structure of the previous patients' brains to stimulation parameters evoking side effects in the previous patients, and
using the accumulated data and the imaging data to determine stimulation parameters for the subject patient.

2. The method of claim 1, wherein the imaging data for the subject patient comprises preoperative magnetic resonance imaging (MRI) data and postoperative computed tomography (CT) and/or MRI data.

3. The method of claim 1, wherein the at least one anatomical feature of the subject patient's brain structure comprises one or more of a medial/lateral axis, an anterior/posterior axis, a medial border, a lateral border, an anterior border, and a posterior border of the brain structure.

4. The method of claim 1, wherein using the imaging data to determine a position of at least one of the electrode leads with respect to at least one anatomical feature of the subject patient's brain structure comprises using the imaging data to determine a 3-D model of the patient's brain structure and voxelizing the 3-D model.

5. The method of claim 4, further comprising using principal component analysis to determine one or more axes of the subject patient's brain structure using the 3-D model.

6. The method of claim 1, wherein the accumulated data comprises indications of stimulation field model (SFM) radii corresponding to stimulation parameters providing therapeutic benefits in at least some of the previous patients.

7. The method of claim 1, wherein determining stimulation parameters for the subject patient comprises:
receiving information indicative of a trial stimulation parameter set for the subject patient,
determining an SFM for the trial stimulation parameter set,
determining a SFM radius for the trial stimulation parameter set, and
comparing the SFM radius for the trial stimulation parameter set to SFM radii from the accumulated data.

8. The method of claim 7, wherein the trial stimulation parameter set is automatically suggested using a stimulation optimization algorithm.

9. The method of claim 1, wherein using the accumulated data and the imaging data to determine stimulation parameters for the subject patient comprises:
displaying on a graphical user interface (GUI):
a representation of a search space indicative of potential trial stimulation parameter sets, and
one or more likelihood maps derived from the accumulated data, wherein the one or more likelihood maps indicate trial stimulation parameter sets within the search space that are likely to evoke side effects in the subject patient.

10. The method of claim 9, wherein the one or more likelihood maps indicate SFM radii corresponding to trial stimulation parameter sets that are likely to evoke side effects for the subject patient.

11. A system for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the system comprising:
an external computing device comprising control circuitry configured to perform a method, the method comprising:
receiving imaging data for the subject patient,
using the imaging data to determine a position of at least one of the electrode leads with respect to at least one anatomical feature of a brain structure the subject patient's brain,
receiving accumulated data from a database, wherein the accumulated data comprises data from previous patients relating electrode lead position with respect to anatomical features of the previous patients' brain structures to stimulation parameters evoking side effects in the previous patients, and
using the accumulated data and the imaging data to determine stimulation parameters for the subject patient.

12. The system of claim 11, wherein the imaging data for the subject patient comprises preoperative magnetic resonance imaging (MRI) data and postoperative computed tomography (CT) and/or MRI data.

13. The system of claim 11, wherein the at least one anatomical feature of the subject patient's brain structure comprises one or more of a medial/lateral axis, an anterior/posterior axis, a medial border, a lateral border, an anterior border, and a posterior border.

14. The system of claim 11, wherein using the imaging data to determine a position of at least one of the electrode leads with respect to at least one anatomical feature of the subject patient's brain structure comprises using the imaging data to determine a 3-D model of the patient's brain structure and voxelizing the 3-D model.

15. The system of claim 14, wherein the method further comprises using principal component analysis to determine one or more axes of the subject patient's brain structure using the 3-D model.

16. The system of claim 11, wherein the accumulated data comprises indications of stimulation field model (SFM) radii corresponding to stimulation parameters providing therapeutic benefits in at least some of the previous patients.

17. The system of claim 11, wherein determining stimulation parameters for the subject patient comprises:
receiving information indicative of a trial stimulation parameter set for the subject patient,
determining an SFM for the trial stimulation parameter set,
determining a SFM radius for the trial stimulation parameter set, and
comparing the SFM radius for the trial stimulation parameter set to SFM radii from the accumulated data.

18. The system of claim 17, wherein the trial stimulation parameter set is automatically suggested using a stimulation optimization algorithm.

19. The system of claim 11, wherein using the accumulated data and the imaging data to determine stimulation parameters for the subject patient comprises:
displaying on a graphical user interface (GUI):
a representation of a search space indicative of potential trial stimulation parameter sets, and
one or more likelihood maps derived from the accumulated data, wherein the one or more likelihood maps indicate trial stimulation parameter sets within the search space that are likely to evoke side effects in the subject patient.

20. The system of claim 19, wherein the one or more likelihood maps indicate SFM radii corresponding to trial stimulation parameter sets that are likely to evoke side effects for the subject patient.
